(19) United States
(12) Patent Application Publication
(10) Pub. No.: US 2011/0075796 A1
(43) Pub. Date: Mar. 31, 2011

(54) ROTARY POWER TRANSFORMER FOR USE IN A HIGH-VOLTAGE GENERATOR CIRCUITRY FOR INDUCTIVELY TRANSMITTING TWO OR MORE INDEPENDENTLY CONTROLLABLE SUPPLY VOLTAGES TO THE POWER SUPPLY TERMINALS OF A LOAD

(75) Inventors: Christoph Loef, Aachen (DE); Peter Luerkens, Aachen (DE)

(73) Assignee: KONINKLIJKE PHILIPS ELECTRONICS N.V., Eindhoven (NL)

(21) Appl. No.: 12/994,474

(22) PCT Filed: May 27, 2009
(86) PCT No.: PCT/IB09/52217

§ 371 Date: Nov. 24, 2010

(30) Foreign Application Priority Data
Jun. 2, 2008 (EP) 08104201.2
Jun. 4, 2008 (EP) 08104248.3

(51) Int. Cl. A61B 6/00; H01F 38/18; H05G 1/10

(52) U.S. Cl. 378/15; 336/118; 378/111

(57) ABSTRACT

The present invention refers to a high-voltage power supply circuit for inductively transmitting electrical energy from a stationary part to a load on a rotary part which requires a non-symmetrical voltage transfer, for example, an X-ray tube of an X-ray computed tomography device. The circuit may be realized as a resonant-type power converter circuit with a single rotary power transformer (500) or more than one such power transformer, where at least two separate DC/AC power inverter stages provide two individually controllable AC input voltages (U1, U2) to different windings (511, 512) of a multi-primary coil belonging to the rotary power transformer. Two output voltages supplied by the multi-secondary coil (521, 522, 523, 524) of said transformer which are derived from the two individually controllable AC input voltages are fed to the tube electrodes for powering the X-ray tube.

The present invention refers to high-voltage generator circuitries that may e.g. be used to supply an electrical power needed for operating an X-ray tube of an X-ray computed tomography device or any other type of load which requires a non-symmetrical voltage transfer to its supply electrodes. More particularly, the present invention is directed to a high-voltage power supply circuit and control method for operating such a power supply circuit which focuses on inductively transmitting electrical energy from a stationary to a rotary part, i.e. from a stationary voltage source via a contactless high power rotary transformer to the electrodes of an X-ray tube of the rotary anode disk type or to a rotary bearing assembly of a CT scanner gantry. The above-mentioned high-voltage power supply circuit may thereby be realized as a resonant-type power converter circuit with a single rotary power transformer or more than one such power transformer post-connected to at least one high-voltage transformer or at least one series resonant tank circuit serially coupled to the respective output ports of at least two separate DC/AC power inverter stages, wherein said power inverter stages serve to provide two individually controllable AC input voltages to different windings of a multi-primary coil belonging to the rotary power transformer. After being rectified and smoothed by at least two separate peak-type rectifiers coupled to different windings of a multi-secondary coil belonging to the rotary power transformer, two output voltages supplied by the multi-secondary coil of said transformer which are derived from the two individually controllable AC input voltages are fed to the tube electrodes for powering the X-ray tube.

### BACKGROUND OF THE INVENTION

High-voltage generators for X-ray tube power supplies as used in medical X-ray imaging typically comprise at least one high-voltage transformer which provides the required power for operating the X-ray tube to the tube's anode and cathode. In conventional high-voltage generators circuits, an AC supply voltage adjusting device, such as e.g. an autotransformer, supplies line power to the multi-phase primary of a high-voltage transformer. A switching device, such as e.g. a semiconductor switch in conjunction with a bridge rectifier, opens and closes the star point of the multi-phase primary to turn on and off high voltage at the X-ray tube. Inductive and capacitive effects in the transformer and associated power supply components generally cause the high voltage to rise above its steady-state level during a period immediately following completion of the circuit. Especially phase-shifted pulse width modulation (PWM) inverter-fed DC/DC power converters with a high-voltage transformer parasitic resonant link as used for an X-ray power generator thereby exhibit stiff nonlinear characteristics due to phase-shifted voltage regulation and diode cutoff operation in a high-voltage rectifier because of the wide load setting ranges in practical applications.

Recently, a variety of switched-mode high-voltage DC power supplies using voltage-fed-type or current-fed-type high-frequency transformer resonant inverters with MOS gate bipolar power transistors (IGBTs) have been developed for medical-use X-ray high power generators.

### SUMMARY OF THE INVENTION

When powering X-ray tubes which comprise a metal envelope and in which the cathode current has to be different from the anode current, a non-symmetrical power distribution to the anode and cathode of the tube is required. Therefore, the two voltages applied to the X-ray tube must be independently controllable.

Whereas the prior art proposes different solutions for providing energy transfer to a load mounted to a stationary part of a system to be operated, some solutions have also been developed for transferring electrical energy to the supply terminals of a load which is either a part of or fixedly attached to a rotatably mounted system assembly.

In known applications as known from the prior art, slip rings are used for making an electrical connection through a rotating assembly, wherein brushes of such a slip ring may be used to transmit high voltages to a load. However, with respect to the mechanical design and construction of the brushed used, the transferable current and rotational speed of the disk is usually limited.

For example, DE 103 56 109 A1 describes a method for transferring electrical energy to a rotating gantry. The herein proposed system is equipped with a rotating part that comprises at least an X-ray tube and a detector arrangement as well as a stationary part. A bearing is provided for the rotational mounting of the rotating part and at least an inverter for generating an alternating current at a first frequency. The stationary part comprises at least a conductor arrangement that is supplied with an AC current from the inverter, while the rotating part has an inductive coupler which meshes with the conductor arrangement in a position dependent manner and couples electrical energy from it. Unfortunately, the system described in DE 103 56 109 A1 is not usable for high frequency operation since the inductances in the winding of the applied transformer will show high values.

In U.S. Pat. No. 5,731,968 A, an X-ray apparatus is described which comprises a power supply section for powering an X-ray tube with a high-voltage transformer equipped with two groups of primary and secondary windings provided on the same transformer core. According to the invention as proposed by U.S. Pat. No. 5,731,968 A, it is foreseen that the coupling between the primary windings belonging to different groups is weaker than the coupling between primary and secondary windings belonging to the same group, wherein the primary windings of the two groups are connected to two inverters which operate at the same frequency. Control of the power at the secondary side can be improved in that the inverters are operated at a fixed frequency and with a duty cycle which can be independently controlled.

It may thus be an object of the present invention to provide a low-weight solution for a high-voltage power supply circuitry which allows to contactlessly empower an X-ray tube in a CT system of the rotating gantry type with two independently controllable tube voltages over a single rotary power transformer core and thus by using a minimized number of transformer coils.

To address this object, a first exemplary embodiment of the present invention is dedicated to a contactless high power rotary transformer for supplying at least two individually controllable AC supply voltages from at least two separate power supplies to at least one load via a single transformer core. The proposed transformer may be equipped with multiple stationarily mounted primary coils serially arranged in at least two non-overlapping sections of a first annular member and multiple secondary coils subsequently arranged in adjacent sections of a second annular member, contactlessly rotatable about a center of rotation which coincides with the centers of the first and second annular members and being inductively coupled via said transformer core to said primary coils. According to this embodiment, each of said primary coils is supplied with a different one of said individually controllable AC supply voltages, and the power supplying electrodes of said load are supplied with different output voltages which are derived from multiple individually controllable AC supply voltages.

In a situation where the secondary coils (of said second annular member are in a rotated position after being shifted by a rotational angle relative to the stationarily mounted primary coils of said first annular member, said transformer may advantageously be adapted to provide at least two different output voltages which are given by a linear combination of at least two individually controllable AC supply voltages fed to the stationarily mounted primary coils of said first annular member.

According to a further aspect of this first exemplary embodiment, it may be foreseen that the weighting coefficients of the aforementioned linear combination may be given by at least two stepwise linear continuous functions of the rotational angle.

For example, said weighting coefficients may be given by two periodically repeated triangular functions of the rotational angle which, in angular direction, are shifted against each other by an offset angle. Pursuant to a special refinement of this embodiment, said functions may both have a minimum value of zero and a maximum height of one. Furthermore, said functions may have the same slope factors in their periodically repeated monotonously inclining and monotonously declining sections. According to the herewith described example, said functions may be shifted by an offset angle of 90° against each other such that a first one of these functions takes on a maximum value when a second one of these functions takes on its minimum value, and vice versa.

A second exemplary embodiment of the present invention is directed to a high-voltage power supply circuitry for supplying an electrical power to a load as given by an X-ray tube of an X-ray computed tomography device which requires a non-symmetrical voltage supply to the power supplying electrodes of said X-ray tube. According to this embodiment, said circuitry may comprise a contactless high power rotary transformer as set forth with reference to said first exemplary embodiment which, at its primary side, may be supplied with a different one of at least two individually controllable AC supply voltages. The power supplying electrodes of said X-ray tube may thereby be supplied with at least two different output voltages which are derived from the aforementioned at least two individually controllable AC supply voltages.

In particular, it may be foreseen that said high-voltage power supply circuitry is realized as a resonant-type power converter circuit with said power transformer being post-connected to at least one high-voltage transformer or at least one series resonant tank circuit serially coupled to the respective output ports of at least two separate DC/AC power inverter stages, wherein the latter serves to provide said at least two individually controllable AC input voltages to different primary coils of said contactless high power rotary transformer's first annular member.

Finally, a third exemplary embodiment of the present invention refers to a high-voltage power supply circuitry for supplying an electrical power to a load such as given by an X-ray tube of an X-ray computed tomography device which requires a non-symmetrical voltage supply to the power supplying electrodes of said X-ray tube, said circuitry comprising more than one contactless high power rotary transformers which, at their primary sides, are each supplied with a different one of at least two individually controllable AC supply voltages. According to this embodiment, said power transformers are each equipped with a stationarily mounted ring-shaped primary coil and a ring-shaped secondary coil contactlessly rotatable about a center of rotation which coincides with the centers of the ring-shaped primary and secondary coils and being inductively coupled via a single transformer core to said ring-shaped primary coil, and the power supplying electrodes of said X-ray tube are supplied by the secondary coils of said power transformers with at least two different output voltages which are derived from the aforementioned at least two individually controllable AC supply voltages.

It can thus be summarized that the circuit as disclosed in the scope of the present application focuses on the operation with high frequency and providing a voltage control for two or more independently controllable supply voltages which are to be fed to an X-ray tube's anode and cathode, respectively, wherein X-ray tubes having a metal envelope can be supplied with electrical power over a single rotary power transformer. Using a single rotary power transformer as proposed by the present invention will benefit in lower cost, weight and size of the required high-voltage generator circuitry, especially when operating at higher frequencies. In contrast to the present application, conventional systems as known from the prior art do not have a capability to transmit two or more independently controllable voltages over a single rotary power transformer.

Typically, an X-ray tube needs different voltages at the cathode and the anode. Therefore, there is a need for different voltages on the rotary part of the gantry. Also for other applications, such as image processing and data transfer, which are arranged at the rotary side of the gantry, it is useful to provide different voltages. Preferably, these different voltages at the secondary side of the transformer are galvanically isolated. Therefore, it is an object of the present invention to provide different galvanically isolated voltages at the secondary side of the transformer.

The present invention arrives at that goal by an inventive arrangement of the windings of the power transformer, which transfers the electrical energy from the stationary part of the gantry to the rotary part of the gantry. Especially, it is provided an arrangement, which comprises two primary windings. Further, according to the invention it is provided an embodiment, which has a first number of primary windings and a second number of secondary windings, wherein the second number is a multiple of the first number. Therefore, as an special embodiment of the invention it is provided a power transformer with two windings on the primary side and with two, four, eight, or with 2 ⁿ windings on the secondary side of the transformer, wherein n is an integer number.

According to the invention it is provided the possibility to couple the different AC voltages, which are generated on the secondary side of the transformer because of the different secondary windings. Especially, it is provided an arrangement of coupling the different AC voltages on the secondary side of the transformer after rectifying the single AC voltages of the single secondary windings. Preferably, it is provided an embodiment, wherein the rectified AC voltages are added in such a way, that there is an increasing of the resulting DC voltage. This leads to the effect, that the necessary cascade, which is arranged at the rotary side of the gantry can be downsized with respect to other embodiments. As a result thereof the rotary part of the gantry will become cheaper and less complicated with respect to the manufacturing.

### BRIEF DESCRIPTION OF THE DRAWINGS

These and other advantageous features and aspects of the invention will be elucidated by way of example with respect to the embodiments described hereinafter and with respect to the accompanying drawings. Therein,

FIG. 1 shows a block diagram for illustrating the principle components of multi-pulse high-voltage generators as commonly used according to the prior art for providing a supply voltage for an X-ray tube,

FIG. 2 shows a closed-loop control circuit for illustrating the principle of X-ray tube voltage and tube current control as known from the prior art,

FIG. 3 shows an analog implementation of an inverter-type high-voltage generator according to the prior art as described with reference to FIG. 1 which may be used in a medical X-ray system,

FIG. 4 shows an analog circuitry of a resonant DC/DC power converter circuit for supplying an output power for use in a high-voltage generator circuitry with two independent DC/AC power inverter stages as known from WO 2006/114719 A1,

FIG. 5 shows an initial position of a rotary transformer as used in the scope of the present invention,

FIG. 6 shows the rotary transformer of FIG. 5 in a rotated position,

FIG. 7 shows weighting functions f and g as used in equations (1) to (4),

FIG. 8 shows a rotary transformer according an embodiment of the present invention with auxiliary difference transformers and rectifiers,

FIG. 9 shows a system configuration of a high-voltage generator circuitry according to the present invention with two separate rotary power transformers, and

FIG. 10 shows a system configuration of a high-voltage generator circuitry according to the present invention with a single rotary power transformers.

### DETAILED DESCRIPTION OF THE PRESENT INVENTION

In the following sections, an exemplary embodiment of the claimed DC/DC power converter circuit as well as an exemplary embodiment of the claimed control method according to the present invention will be explained in more detail, thereby referring to the accompanying drawings.

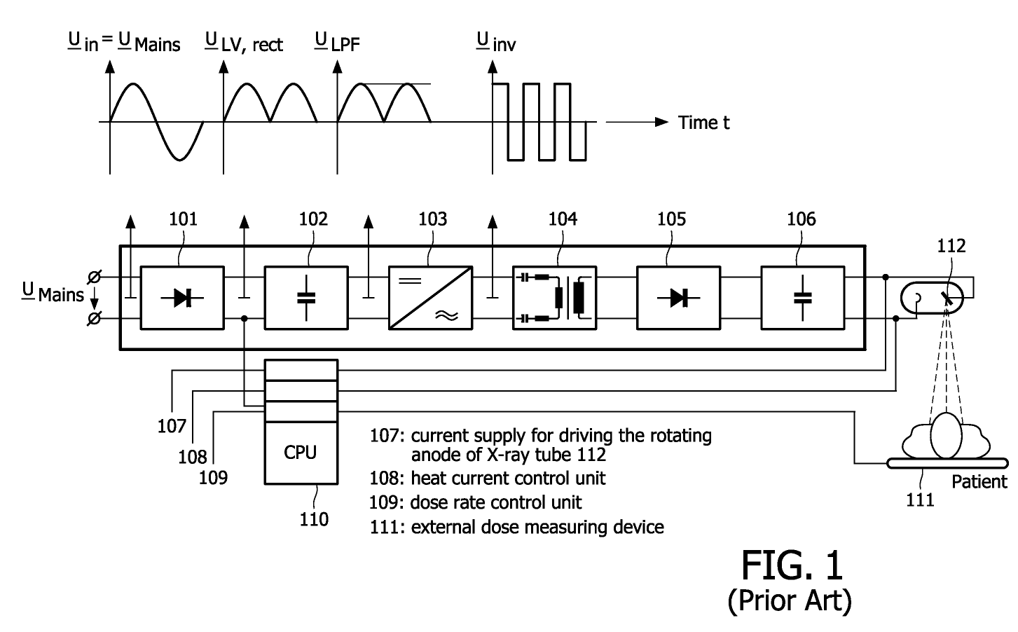

FIG. 1 illustrates the principle of high-frequency inverter technology, which is also known as direct voltage conversion. It thereby shows the principle components of a conventional multi-pulse high-voltage generator used for providing the supply voltage of an X-ray tube 112. First, an intermediate DC voltage U_{LPF} with more or less ripple is generated by rectifying and low-pass filtering an AC supply voltage U_{Mains} which is supplied by the mains, thereby using an AC/DC converter stage 101 followed by a first low-pass filtering stage 102, wherein the latter may simply be realized by a single smoothing capacitor. Although the electric output power will naturally differ, the same high-voltage quality can be obtained from a single-phase power source as from a three-phase power source. A DC/AC power inverter stage 103 post-connected to said low-pass filtering stage 102 then uses the intermediate DC voltage to generate a high-frequency alternating voltage Uᵢₙᵥ, feeding a dedicated high-voltage transformer 104 which is connected on its secondary side to a high-voltage rectifier 105 and a subsequent second low-pass filtering stage 106, wherein the latter may also be realized by a single smoothing capacitor. The obtained output voltage Uₒᵤₜ may then be used as a high-frequency multi-pulse tube voltage for generating X-radiation in the X-ray tube 112.

In this context, it should be noted that high-frequency inverters normally apply pulse-width modulation or act as a resonant circuit type depending on the power switches used. On the assumption that the depicted multi-pulse high-voltage generator circuitry allows for a reduction in the transformer core cross section, transformation of high-frequency AC supply voltages yields a very small high-voltage transformer volume. With such a circuitry, X-ray tube voltage and current can be controlled independently and are largely unaffected by power source voltage fluctuations. Electronic X-ray tube voltage control units thereby typically exhibit a response time of 0.1 ms or less.

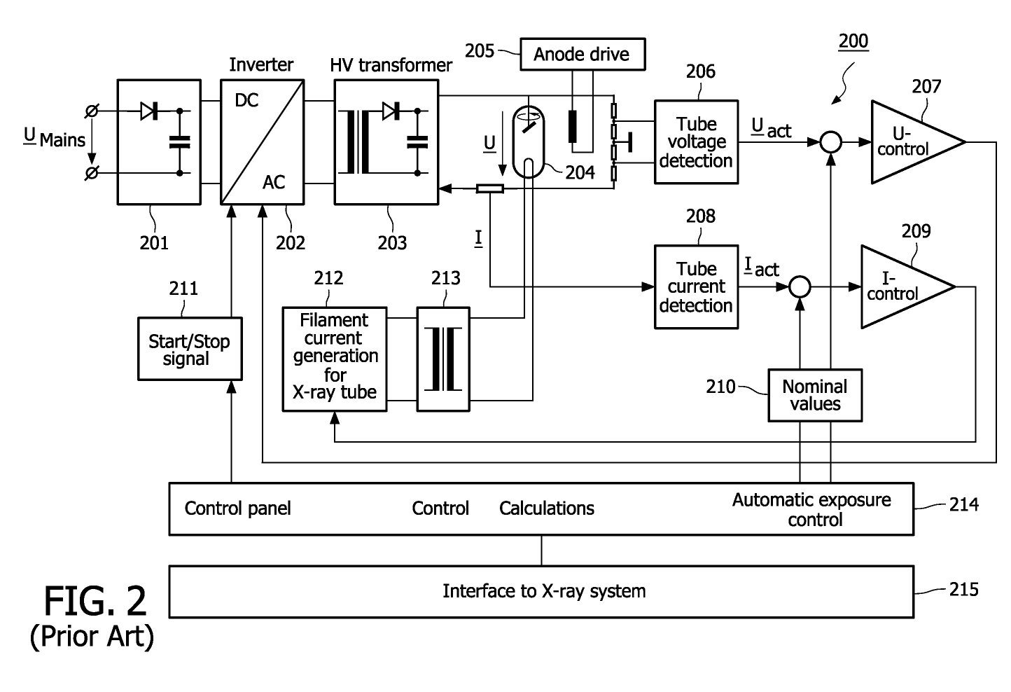

A closed-loop control circuit for illustrating the principle of X-ray tube voltage and tube current control as known from the prior art is shown in FIG. 2. Typically, an actual value U_{act} of X-ray tube voltage is measured and compared to a nominal value Uₙₒₘ, selected by the operator at the control console in a comparator circuit. Depending on this information, the power switches are adjusted in a predefined manner (such as e.g. described in WO 2006/114719 A1). The speed of this control depends mainly on the inverter frequency. Although it is not quite as fast as constant potential high-voltage generators, the inverter easily exceeds the speed of conventional multi-peak rectifiers. The ripple in the resulting voltage on the secondary side of the transformer is influenced mainly by the inverter frequency, the internal smoothing capacity, the capacity of the high-voltage supply cables and the level of the intermediate DC voltage U_{LPF}.

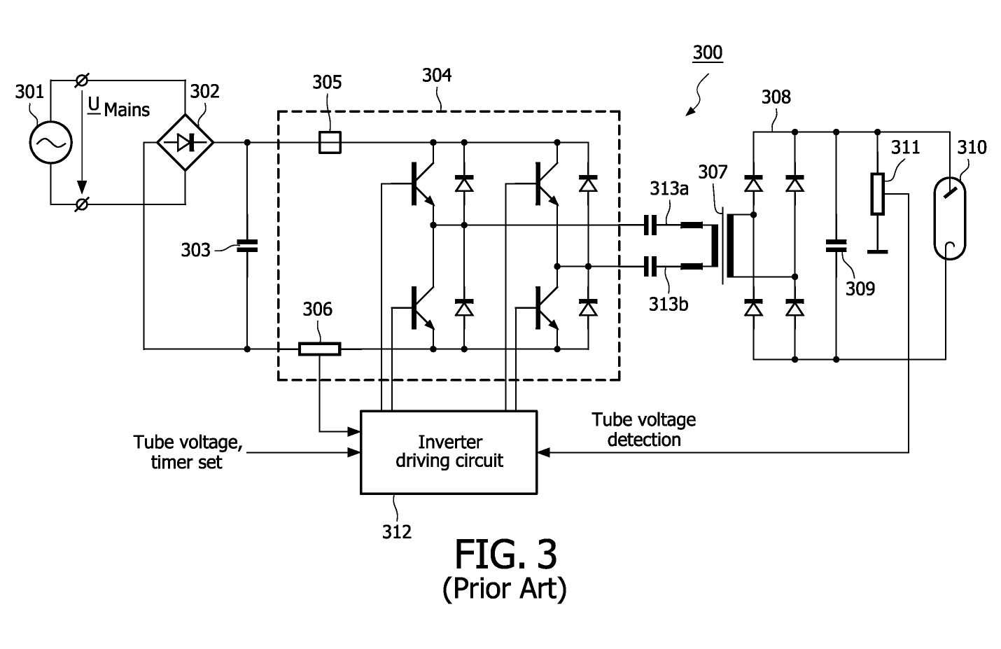

An analog implementation of an inverter-type high-voltage generator according to the prior art as described with reference to FIG. 1, which may e.g. be used in a medical X-ray system, is shown in FIG. 3. As shown in FIG. 3, an AC supply voltage supplied from the mains is rectified and smoothed by a full-wave rectifier 302 and a smoothing capacitor 303 into an intermediate DC voltage and then supplied to a DC/AC full-bridge power inverter stage 304 consisting of four bipolar high-power switching transistors. Furthermore, a fuse 305 is connected to one end of the input side of the inverter circuit 304, and a current detector 306 is connected to the other end of the inverter circuit 304.

First, a DC input voltage is converted into a high-frequency AC supply voltage (e.g., 200 kHz) by means of inverter circuit 304. After that, said AC supply voltage is transformed into an AC supply voltage of a higher level (e.g., 150 kV) by means of a high-voltage transformer 307 which is then rectified and smoothed by a high-voltage rectifier 308 and a smoothing capacitor 309. Said high-voltage rectifier 308 may be given by a silicon rectifier with a breakdown voltage of about 150 kV, etc. Finally, the obtained DC high voltage is applied to an X-ray tube 310. A voltage dividing resistor 311 is connected in parallel with the capacitor 309. As a detection value of a tube voltage (i.e., a detection value corresponding to the applied voltage to the X-ray tube), a voltage across the voltage dividing resistor 311 is fed back to an inverter driving circuit 312 which is used for controlling the switching timing of the inverter circuit 304.

To the inverter driving circuit 312, a detection value of the inverter current detector 306, the detection value of the tube voltage, a set value for setting the tube voltage as well as a set value (exposure time) for setting a timer are fed. These values are respectively input via a console (not shown) of the X-ray system. As depicted in FIG. 3, the inverter driving circuit 312 generates an output signal which drives the switching transistors of the inverter circuit 304.

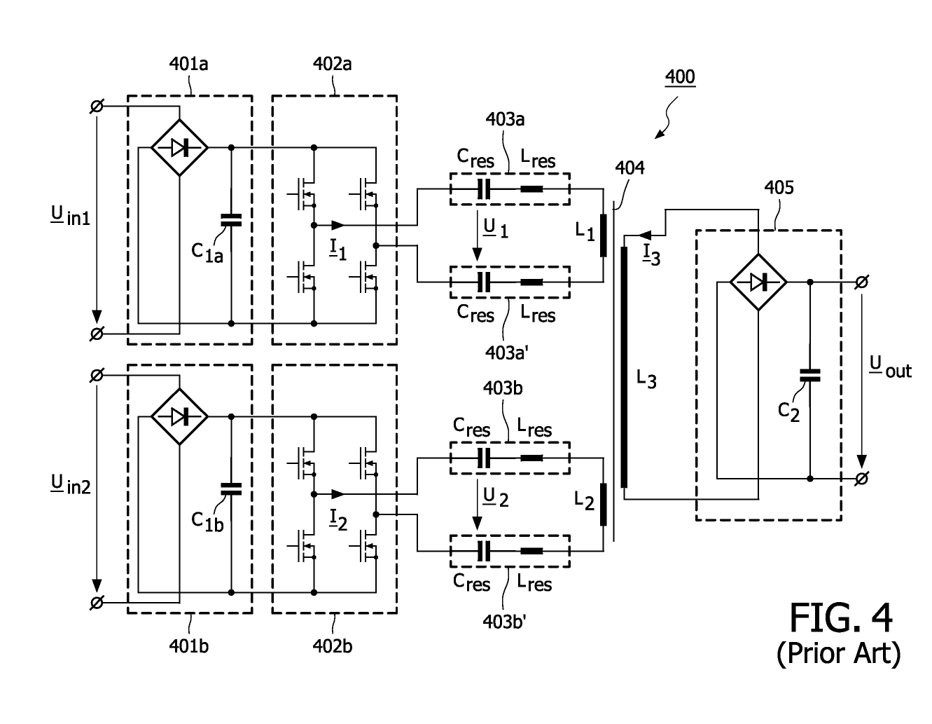

CT or X-ray high-voltage generators preferably consist of DC/AC full-bridge power inverter stages which are connected to a series resonant circuit for driving the high-voltage transformer (cf. FIG. 4). In this figure, an analog circuitry of a resonant DC/DC power converter circuit for supplying an output power for use in a high-voltage generator circuitry with two independent DC/AC power inverter stages as known from WO 2006/114719 A1 is shown. Therein, it is depicted how two inverter circuits 402a+b can work on one high-voltage transformer 404 with multiple windings. It can be shown that the size of discrete steps of the DC/DC power converter output voltage (Uₒᵤₜ can be reduced, resulting in an even lower output voltage ripple. Due to the coupling of the two resonant circuits by the common transformer, a voltage divider function is realized.

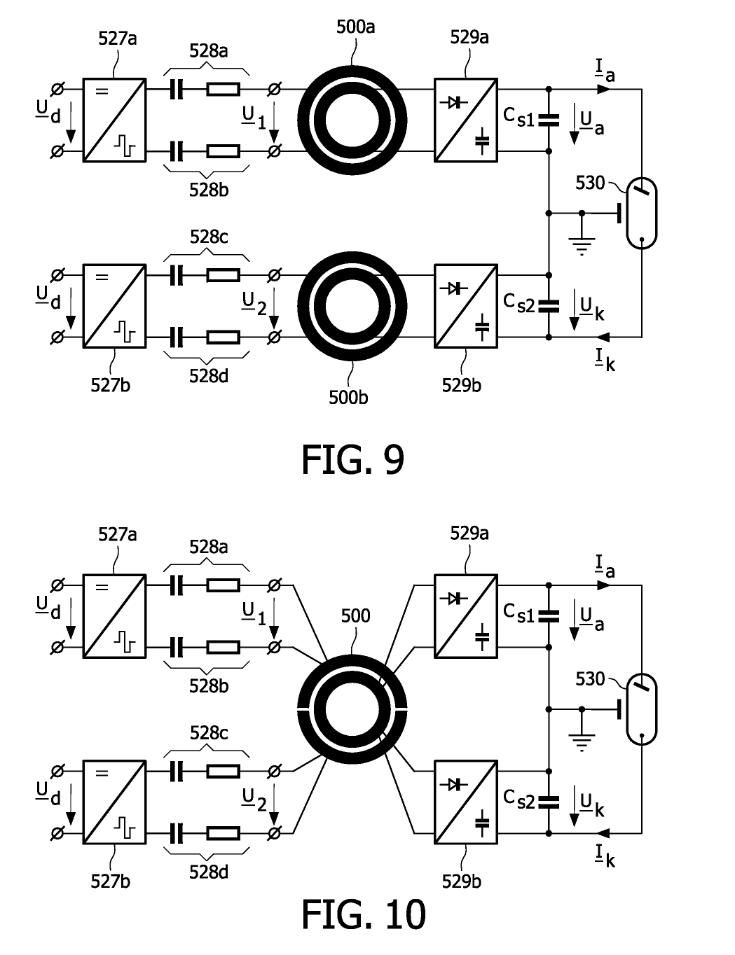

To provide the anode and cathode of an X-ray computed tomography device's X-ray tube with two independently controllable voltages, two separate ring-shaped power transformers of a concentric ring-shaped type as shown in the system configuration depicted in FIG. 9 can be used. The depicted circuitry thereby comprises two independent (contactless) power transformers 500a and 500b of the rotary type, which hence results in a large volume and weight of the entire circuitry. According to this embodiment, the primary coil of the rotary power transformer is connected in series to two series resonant tank circuits placed at the respective output ports of two DC/AC power inverter stages, wherein said inverter stages are used for providing independently controllable voltages Uₐ and Uₖ to the rotary power transformer. After being fed to a corresponding one of two peak-type rectifiers 529a and 529b post-connected to the secondary coil of power transformers 500a and 500b, respectively, a rectified and smoothed version of said voltages (Uₐ and Uₖ respectively) is stored in a respective one of two storage capacitors (C_{S1} or C_{S2}) connected to an X-ray tube's anode and to the ground electrode of the circuitry or to the X-ray tube's cathode and to the ground electrode, respectively, and supplied to the electrodes of said X-ray tube (the latter being referred to by reference number 530) which requires a non-symmetrical power distribution to its anode and cathode.

Alternatively, a circuit configuration as depicted in FIG. 10 which requires less size and weight can be designed. As shown in FIG. 10, this circuitry merely uses a single rotary power transformer 500 of the concentric ring-shaped type comprising a dedicated winding configuration. Furthermore, two DC/AC power inverter stages 527a and 527b are foreseen which provide two AC output voltages to supply the high voltage generator on the rotating gantry.

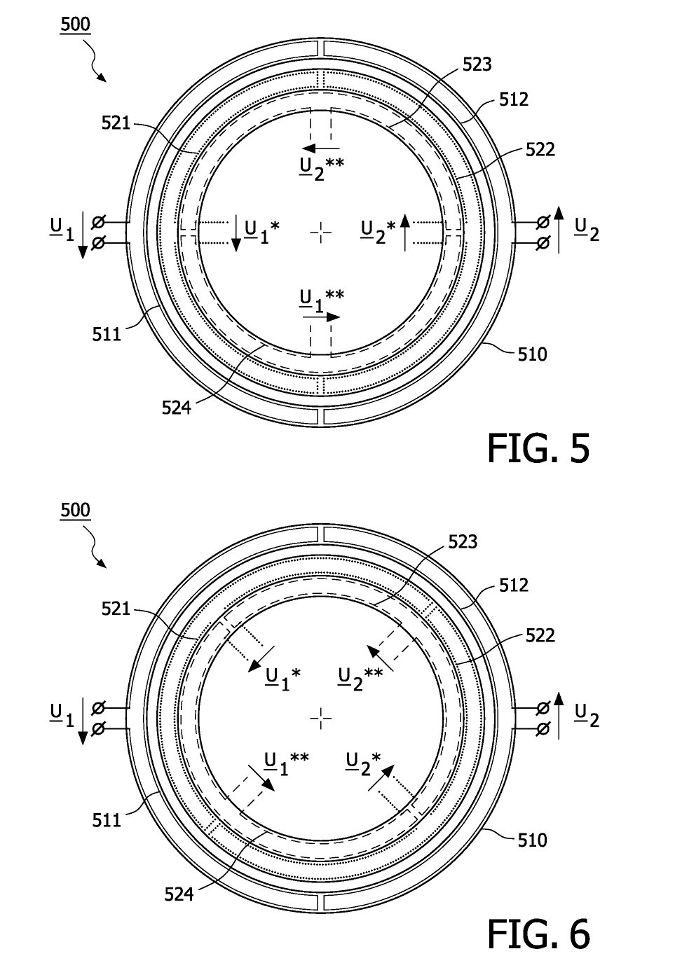

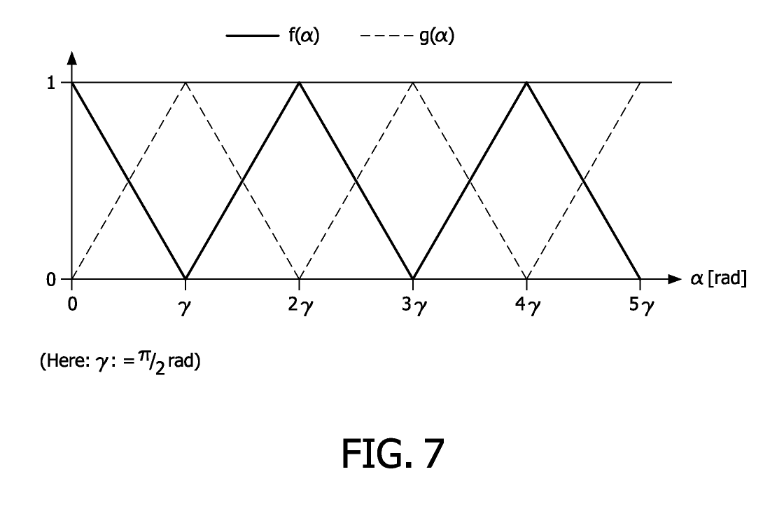

In the following, the winding configuration and the corresponding wiring of the proposed rotary power transformer will be described with reference to FIGS. 5 and 6. As shown in these figures, the objective of the invention is achieved by dividing the primary and the secondary part of power transformer 500 into a number of separate sections. The primary part 510, herein also referred to as first annular member, comprises at least two primary coils or sections (511 and 512) for keeping AC input voltages U₁ and U₂ supplied by two separate AC power sources (mot shown) independent of each other. The secondary part 520, also referred to as second annular member, is realized in the form of two concentric rings each consisting of two sections (521, 522 and 523, 524, respectively) whose size division ratio corresponds to the size division ratio of the first annular member. The inner ring of the two concentric rings is rotated by half of the sectional pitch.

FIG. 5 shows rotary transformer 500 in an initial position. As described above, the primary part 510 constituting said first annular member comprises two primary coils 511 and 512 forming two sections. The secondary part 520 constituting said second annular member comprises four secondary coils forming a first set of sections (521, 522) belonging to the outer ring of the second annular member and a second set of sections (523, 524) belonging to the inner ring of said second annular member, wherein the inner ring is shifted by an offset angle of 90° relative to said outer ring.

In this position, AC output voltage U₁^{*} picked up from first secondary coil 521 of said second annular member's outer ring corresponds to AC input voltage U₁ of first primary coil 511, whereas AC output voltage U₂^{*} picked up from second secondary coil 522 of said second annular member's outer ring corresponds to AC input voltage U₂ of second primary coil 512. At the same time, AC output voltage U₁^{**} corresponds to a weighted superposition of AC input voltage U₁ at first primary coil 511 and AC input voltage U₂ at second secondary coil 512. The same applies to AC output voltage U₂^{**}. The weighting coefficients in the linear combination of the superimposing components can be expressed as stepwise linear functions f and g of the overlapping angle. In particular, when secondary annular member 520 is rotated as a whole by a rotational angle of 90° , the properties of AC output voltages U₁^{*} and U₁^{**} and the properties of AC output voltages U₂^{*} and U₂^{**} are exchanged.

A simplified mathematical representation of this relationship which does not consider weak potential couplings of the said first (511) and second primary coil (512) of first annular member 510 can be expressed by the following equations yielding the voltage drops U₁^{*} and U₂^{*} across first (521) and second (522) secondary coil of said second annular member's outer ring:

U₁^{*}=U₁·f(α)+U₂·g(α), (1)

U₂^{*}=U₁·f(α+γ)+U₂·g(α+γ). (2)

Therein, a denotes a rotational angle by which secondary coils 521, 522, 523 and 524 of said second annular member 520 are shifted relative to the stationarily mounted primary coils 511 and 512 of said first annular member 510, and γ denotes an offset angle by which weighting functions f and g, which may advantageously be given as two periodically repeated triangular functions having the same minimum level, slope factors and the same height, are shifted against each other along the direction of an axis labeled with rotational angle α. In the embodiments of rotary power transformer 500 as depicted in FIGS. 5, 6 and 8, γ is given by an offset angle of 90° .

Similarly, the voltage drops across the first (523) and second (524) secondary coil of said second annular member's inner ring can be expressed as follows:

U₁^{**}=U₁·f(α+γ/2)+U₂·g(α+γ/2), (3)

U₁^{**}=U₁·f(α+3γ/2)+U₂·g(α+3γ/2), (4)

Weighting functions f and g can thereby be chosen such that the sum of AC output voltages U₁^{*} and U₂^{*} is constant and has the same value as the sum of AC output voltages U₁ ** and U₂^{**}. Assuming a transfer ratio of 1:1 of the transformer, it can be provided that the average voltage of AC output voltages U₁^{*} and U₂^{*} is equal to the average voltage of AC output voltages U₁^{**} and U₂^{**} as well as to the average voltage (̅U̅₁̅,̅ U̅₂̅)̅=(U₁+U₂)/2 of AC input voltages U₁ and U₂.

The sum of the rectified difference voltage of the two secondary sets U₁^{*} and U₂^{*}, and the sum of voltages U₁^{**} and U₂^{**}, respectively, is two times the absolute difference of the two input voltages:

U₁¹+U₂^{*}=U₁^{**}+U₂^{**}=2·|U₁−U₂|. (5)

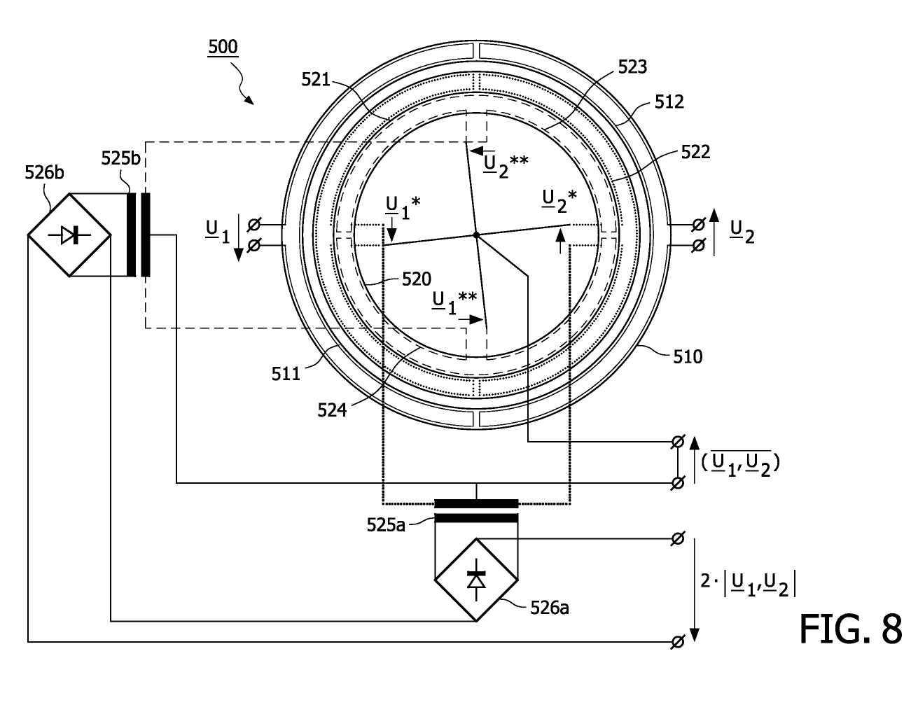

It is therefore possible to generate and obtain two different voltages (U₁^{*} and U₂^{*} or U₁^{**} and U₂^{**}) at the output terminals of rotary power transformer 500 by providing the two primary coils 511 and 512 of said first annular member 510 with two different voltages U₁ and U₂ and connecting the output terminals of the four secondary coils 521, 522, 523 and 524 via the secondary coils of two auxiliary difference transformers 525a and 525b whose primary coils are coupled to the output terminals of two serially connected AC/DC converters 526a and 526b used for rectifying difference voltage 2·|U₁−U₂| which is fed the input port of a first one (526a) of these AC/DC converters (cf. FIG. 8).

In the same way, the initial difference of the input voltage can also be created by connecting difference transformers to the second annular member.

FIGS. 5 and 6 show the arrangement of the primary and secondary windings. It is depicted two primary windings at the primary coils 511 and 512. Further, it is shown two arrangements of secondary windings, wherein there are together four secondary windings.

Applications Of The Invention

The above-described invention can advantageously be applied in the field of high-voltage generator circuitries used for supplying electrical power to a load which requires a non-symmetrical voltage supply at its electrodes such as e.g. for supplying an X-ray tube of an X-ray computed tomography device. Aside therefrom, the invention may also be usefully applied for proceeding the development of power transformer circuit technology in general.

It should be noted that the present invention is not limited to applications which only require to feed two independently controllable supply voltages to two power supply terminals of a load as described with reference to the embodiments depicted in FIGS. 9 and 10, but that it can also be applied in the scope of other applications which require to feed more than two independently controllable supply voltages to any type of load with more than two power supply terminals.

While the present invention has been illustrated and described in detail in the drawings and in the foregoing description, such illustration and description are to be considered illustrative or exemplary and not restrictive, which means that the invention is not limited to the disclosed embodiments. Other variations to the disclosed embodiments can be understood and effected by those skilled in the art in practicing the claimed invention, from a study of the drawings, the disclosure and the appended claims. In the claims, the word “comprising” does not exclude other elements, and the indefinite article “a” or “an” does not exclude a plurality. The mere fact that certain measures are recited in mutually different dependent claims does not indicate that a combination of these measures can not be used to advantage. Furthermore, it should be noted that any reference signs in the claims should not be construed as limiting the scope of the invention.

1. A contactless high power rotary transformer (500) for supplying at least two individually controllable AC supply voltages from at least two separate power supplies to at least one load (530) via a single transformer core, wherein said power transformer is equipped with multiple stationarily mounted primary coils (511, 512) serially arranged in at least two non-overlapping sections of a first annular member (510) and multiple secondary coils (521, 522, 523 and 524) subsequently arranged in adjacent sections of a second annular member (520), contactlessly rotatable about a center of rotation which coincides with the centers of the first and second annular members (510, 520) and being inductively coupled via said transformer core to said primary coils (511, 512), wherein each of said primary coils (511, 512) is supplied with a different one of said individually controllable AC supply voltages (U₁, U₂) and wherein the power supplying electrodes of said load (530) are supplied with different output voltages (Uₐ, Uₖ) which are derived from multiple individually controllable AC supply voltages (U₁, U₂).

2. The contactless high power rotary transformer (500) according to claim 1 which, in a situation where the secondary coils (521, 522, 523 and 524) of said second annular member (520) are in a rotated position after being shifted by a rotational angle (α) relative to the stationarily mounted primary coils (511, 512) of said first annular member (510), is adapted to provide at least two different output voltages (Uₐ, Uₖ) which are given by a linear combination of at least two individually controllable AC supply voltages (U₁, U₂) to the stationarily mounted primary coils (511, 512) of said first annular member (510).

3. The contactless high power rotary transformer (500) according to claim 2, wherein the weighting coefficients of the linear combination are given by at least two stepwise linear continuous functions (f, g) of the rotational angle (α).

4. The contactless high power rotary transformer (500) according to claim 2, wherein the weighting coefficients of the aforementioned linear combination are given by two periodically repeated triangular functions (f, g) of the rotational angle (α) which, in angular direction, are shifted against each other by an offset angle (γ).

5. The contactless high power rotary transformer (500) according to claim 4, wherein said functions both have a minimum value of zero and a maximum height of one.

6. The contactless high power rotary transformer (500) according to claim 5, wherein said functions (f, g) have the same slope factors in their periodically repeated monotonously inclining and monotonously declining sections.

7. The contactless high power rotary transformer (500) according to claim 6, wherein said functions (f, g) are shifted by an offset angle of 90° against each other such that a first one of these functions (f) takes on a maximum value when a second one of these functions (g) takes on its minimum value, and vice versa.

8. A high-voltage power supply circuitry for supplying an electrical power to a load as given by an X-ray tube (530) of an X-ray computed tomography device which requires a non-symmetrical voltage supply to the power supplying electrodes of said X-ray tube, said circuitry comprising a contactless high power rotary transformer (500) according to claim 1 which, at its primary side, is supplied with a different one of at least two individually controllable AC supply voltages (U₁, U₂) and wherein the power supplying electrodes of said X-ray tube (530) are supplied with at least two different output voltages (Uₐ, Uₖ) which are derived from the aforementioned at least two individually controllable AC supply voltages (U₁, U₂).

9. The high-voltage power supply circuitry according to claim 8, realized as a resonant-type power converter circuit with said power transformer (500) being post-connected to at least one high-voltage transformer (525a+b) or at least one series resonant tank circuit (528a-d) serially coupled to the respective output ports of at least two separate DC/AC power inverter stages (527a+b), the latter serving to provide said at least two individually controllable AC input voltages to different primary coils (511, 512) of said contactless high power rotary transformer's first annular member (510).

10. The high-voltage power supply circuitry for supplying an electrical power to a load as given by an X-ray tube (530) of an X-ray computed tomography device which requires a non-symmetrical voltage supply to the power supplying electrodes of said X-ray tube, said circuitry comprising more than one contactless high power rotary transformers (500a+b) which, at their primary sides, are each supplied with a different one of at least two individually controllable AC supply voltages (U₁, U₂), wherein said power transformers are each equipped with a stationarily mounted ring-shaped primary coil (510) and a ring-shaped secondary coil (520) contactlessly rotatable about a center of rotation which coincides with the centers of the ring-shaped primary and secondary coils and being inductively coupled via a single transformer core to said ring-shaped primary coil (510) and wherein the power supplying electrodes of said X-ray tube (530) are supplied by the secondary coils (520) of said power transformers (500a+b) with at least two different output voltages (Uₐ, Uₖ) which are derived from the aforementioned at least two individually controllable AC supply voltages (U₁, U₂).

11. An X-ray computed tomography device comprising a rotary gantry rotatably mounted to a stationary bearing assembly, wherein said X-ray computed tomography device additionally comprises a high-voltage power supply circuitry according to claim 8.
